(19) United States
(12) Patent Application Publication
(10) Pub. No.: US 2008/0026040 A1
(43) Pub. Date: Jan. 31, 2008

(54) Active agent-releasing dosage forms

(76) Inventors: Isaac Farr, Corvallis, OR (US); Leslie Rivera Guzman, Carolina, PR (US); Ricardo G. Diaz-Felipe, Camas, WA (US); Javier Valentin-Sivico, Aguadilla, PR (US); Saul Tirado, Isabela, PR (US); Iddys D. Figueroa, Dorado, PR (US); Kevin Michael Kane, Oakland Park, FL (US); Mirayda Aponte, Aguadilla, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/496,770

(22) Filed: Jul. 31, 2006

(51) Int. Cl. A61K 9/70; A61K 31/18; A61P 43/00

(52) U.S. Cl. 424/443; 514/603

(57) ABSTRACT

The present invention provides pharmaceutical compositions. In one aspect, a pharmaceutical composition is provided having a plurality of polymeric film layers heat sealed together as a multilayer structure and having an active agent dispersed within the multilayer structure. The multilayer structure is configured to release the active agent upon administration to a subject, either in a controlled release or immediate release manner.

### BACKGROUND OF THE INVENTION

Controlled release pharmaceuticals have become very important in the treatment of many medical conditions. Such controlled release formulations have in fact, been found to be desirable in treating many chronic conditions, such as chronic pain, that would otherwise require inconvenient multiple daily doses. Additionally, controlled release dosage forms tend to maintain more consistent blood serum levels with less fluctuation, and thus may reduce undesirable side effects. However, oral dosage sustained release formulations are often complicated in their design, and are thus tend to be more expensive for the manufacturer and the consumer than immediate release dosage forms. Further, because of the nature of certain types of drugs, often, it is desirable to modify or carry drugs in specific ways for even immediate release drugs. Thus, improved controlled release formulations and immediate release formulations that provide certain advantages continue to be sought.

### BRIEF DESCRIPTION OF THE DRAWINGS

FIG. 1 is a schematic representation of an embodiment of a polymeric film layer having active agent disposed thereon;

FIG. 2 is a schematic representation of an embodiment of a polymeric film layer having active agent applied in more discrete locations than that of FIG. 1;

FIG. 3 is a schematic representation of an embodiment of a plurality of polymeric film layers that are stacked into a multilayer structure; and

FIG. 4 is a schematic representation of an embodiment of a plurality of polymeric film layers heat sealed around the edges where no active agent or drug is present.

### DETAILED DESCRIPTION OF THE PREFERRED EMBODIMENT(S)

Reference will now be made to exemplary embodiments, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art having possession of this disclosure, are to be considered within the scope of the present invention.

In describing and claiming the present invention, the following terminology will be used in accordance with the definitions set forth below.

The singular forms “a,” “an,” and “the” include plural referents unless the context clearly dictates otherwise. Thus, for example, reference to “a polymer matrix” includes one or more of such materials, reference to “an excipient” includes reference to one or more of such additive, and reference to “the step of” includes reference to one or more of such steps.

As used herein, “active agent” and “drug,” can be used interchangeably to refer to an agent or substance that has measurable specified or selected physiologic activity when administered to a subject in a pharmaceutically effective amount. The active agent can be a therapeutic, a prophylactic, or a diagnostic agent. These terms of art are well-known in the pharmaceutical and medicinal arts.

As used herein “prodrug” refers to a molecule that will convert into a drug or active agent (its commonly known pharmacological active form). Prodrugs themselves can also be pharmacologically active, and therefore are also expressly included within the definition of an “active agent” as recited above.

As used herein, “immediate release” refers to pharmaceutical compositions that are intended to deliver an active agent or drug rapidly. This can vary from one dosage form to another, but typically delivers the active agent to the subject within an hour, and often immediately.

As used herein, “controlled release” refers to any pharmaceutical composition where an active agent or drug delivery profile is modified from immediate drug release. Non-limiting examples of controlled release include sustained release, delayed release, pulsatile release, etc. Controlled release can include dosing that occurs over a period of at least 1-2 hours, and can often last for a period of days or even weeks. A 12 hour or 24 hour dosing release profile is common.

As used herein, “subject” refers to a mammal that may benefit from the administration of a drug composition or method of this invention. Examples of subjects include humans, and may also include other animals such as horses, pigs, cattle, dogs, cats, rabbits, aquatic mammals, etc.

As used herein, “oral dosage form” and the like refers to a formulation that is ready for administration to a subject via the alimentary canal.

As used herein, “transdermal” refers to the route of administration taken by a drug that is applied to and absorbed through an area of unbroken skin. Thus, the term “transdermal dosage form” refers to formulations or compositions that are applied to a surface of the skin and transdermally absorbed. One example of a transdermal dosage form is a transdermal patch. The term “transdermal patch” refers to a matrix or liquid reservoir type of transdermal delivery device which is used to transdermally deliver defined doses of a drug, over a specific application period.

The term “transmucosal” refers to the route of administration that is taken by a drug that is applied to and absorbed through a mucosal surface. Thus, the term “transmucosal dosage form” refers to a formulation or composition that is applied to a mucosal surface and transmucosally absorbed. Examples of transmucosal dosage forms may include, without limitation, buccal, vaginal, and anal, e.g., patches, suppositories, or other devices.

As used herein, “pharmaceutically acceptable carrier” and “carrier” may be used interchangeably, and refer to any inert and pharmaceutically acceptable material that has substantially no biological activity, and makes up a substantial part of the formulation.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Aspects of the present invention are directed to a pharmaceutical composition, and methods of making and using the composition. It is noted that compositions and methods of the present invention share certain commonalities and characteristics. Some of these characteristics will be discussed in the context of the compositions, and others will be described in the context of the methods. It should be noted that regardless of where discussed, each of the following descriptions applies generally to the pharmaceutical compositions and methods for making and using such pharmaceutical compositions.

Accordingly, in one aspect of the present invention, a pharmaceutical composition can comprise a plurality of polymeric film layers heat sealed together as a multilayer structure; and controlled amounts of active agent dissolved or dispersed in a liquid vehicle which have been ink-jetted on at least one of the plurality of polymeric film layers prior to being heat sealed together, wherein the active agent is within the multilayer structure. In one embodiment, a method of using a pharmaceutical composition can comprise administering the pharmaceutical composition to the subject.

In another embodiment, a method of making a pharmaceutical composition can comprise dispersing or dissolving an active agent in a liquid vehicle to form an ink-jettable composition; precisely ink-jetting the ink-jettable composition on at least one of a plurality of polymeric film layers; and heat sealing the plurality of polymeric film layers into a multilayer structure.

In yet another embodiment, a system for making a pharmaceutical composition can comprise an ink-jetting means for digitally applying an active agent to only a portion of at least one of a plurality of polymeric film layers; and a heat sealing means for bonding the plurality of polymeric film layers into a multilayer structure such that the heat sealing occurs only where no active agent is present in the multilayer structure.

In each of these embodiments, the multilayer structure can be configured to release the active agent upon use in a controlled release manner, or in an immediate release manner. Factors that can affect this include number of layers, materials chosen for use, type of application, e.g., oral, transdermal, transmucosal, parenteral, etc. More specifically, such controlled release or immediate release characteristics may further be a result of the polymeric nature of the polymeric film layers, spatial configuration of the plurality of polymeric film layers, distribution of the active agent within the multilayer structure, characteristics of the active agent such as polarity and size, etc.

In one aspect, for example, a particular controlled vs. immediate release profile of the active agent from the multilayer structure can be accomplished through, inter alia, the compositional nature of the polymeric film layer. It should be noted that the selection of a particular polymeric material for use as a polymeric film layer may also depend on other factors such as the dosage form of the composition. That being stated, release of the active agent can be controlled through utilizing polymeric materials with particular solubilities. For example, a polymeric material that is substantially water soluble will release an active agent into an aqueous environment more rapidly than a slightly water soluble polymer. Active agent release from slightly or from non-water soluble polymers will occur at slower rates due to the diffusion of the active agent from the polymer, as compared to the breakdown of a soluble polymer in an aqueous environment. Similarly, various dosage forms will provide release of the active agent at rates that correspond to the diffusion rate of the active agent through the various polymeric layers of the multilayer structure. Thus different polymer layer combinations may further modify the release characteristics of a given active agent.

The pharmaceutical compositions according to aspects of the present invention can be formulated as a variety of dosage forms, including, but not limited to, oral formulations, transdermal formulations, transmucosal formulations, and the like. As such, the polymeric materials utilized in the multilayer structure may vary according to the dosage form and the intended release characteristics of the active agent. For example, a more water soluble polymeric material will release many active agents more rapidly than a less water soluble polymeric material in an aqueous environment. Additionally, the density of the polymeric material matrix may also play affect release characteristics of the active agent. For example, for two polymeric materials having similar water solubilities but different polymeric densities, the less dense polymeric material will tend to allow diffusion of active agent therefrom at a higher rate as compared to the higher density polymeric material. Accordingly, numerous controlled release profiles can be accomplished through the selection of the polymeric materials from which to construct the polymeric film layers.

In another aspect of the present invention, release of the active agent can be controlled through the spatial configuration of the polymeric film layers within the multilayer structure. Given the differential rate of diffusion of an active agent through different polymeric materials having disparate compositional characteristics, release rate of the active agent may be controlled through the specific relative arrangement of polymeric film layers. For example, multilayer structures having polymeric film layers comprised of the same polymeric material may have a tendency to release active agent at a fairly uniform rate. Multilayer structures having polymeric film layers comprised of different polymeric material, on the other hand, may have a tendency to release active agent at a non-uniform rate. As an example, a multilayer structure having more insoluble or more polymerically dense layers arranged toward the exterior of the structure will tend to delay the release of the active agent contained therein following contact with the aqueous environment. As another example, multilayer structures having more soluble layers toward the exterior and more insoluble layers toward the interior may provide release profiles having early onset of release of a portion of the active agent from the soluble layers followed by a sustained release period as the drug from the more insoluble interior layers diffuse outward. Additionally, alternating the solubilities of adjacent layers may provide a pulsatile-type release of the active agent.

In yet another aspect, release characteristics may be altered by the nature of the heat seal between polymeric film layers of the multilayer structure. For example, designing a defect into the heat seal may be utilized to provide a delayed increase in the rate of release of the active agent following administration. As fluid osmotically enters the sealed multilayer structure it begins to swell. Such swelling may cause a rupture at the defect in the heat seal, thus causing an increase rate of drug release from the formulation.

With respect to the location of the drug, the active agent may be dispersed throughout substantially all of the multilayer structure, or it may be present within only a portion thereof. In one aspect, the active agent can be located substantially between adjacent polymeric film layers. For example, the active agent may be applied to at least one of the plurality of polymeric film layers prior to constructing the multilayer structure, and thus the active agent would be located primarily between adjacent layers rather than dispersed substantially within each layer. It should be noted that the active agent may become dispersed somewhat within a portion of the polymeric film layer depending on the structural characteristics of the active agent and the polymer material making up the film layer.

The controlled release characteristics of a particular pharmaceutical formulation can also be affected by properties of a particular active agent that may affect its diffusion, such as polarity, size, form (i.e. liquid or powder), etc. As such, further control over release can be accomplished through various combinations of active agent and polymeric material.

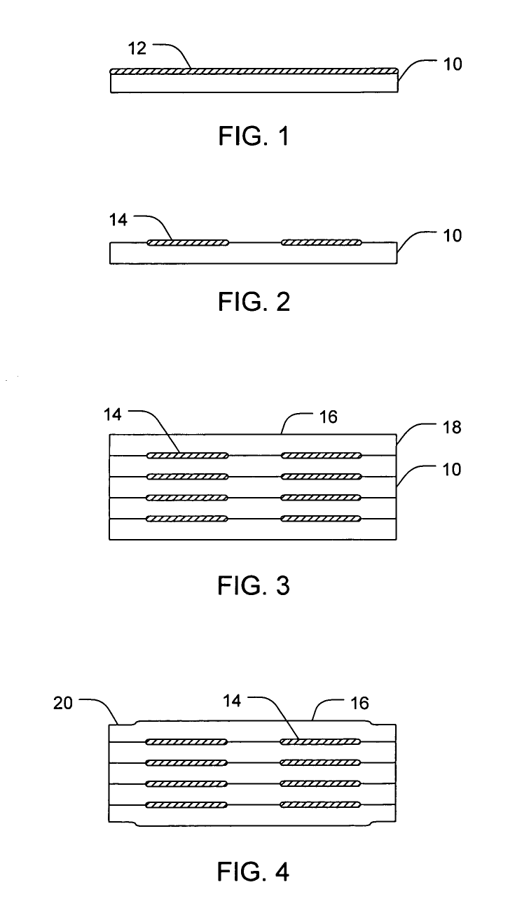

Numerous methods are contemplated for making the multilayer structures of the present invention. In one aspect, a method of making a pharmaceutical composition may include applying an active agent to only a portion of at least one of a plurality of polymeric film layers and bonding the plurality of polymeric film layers into a multilayer structure. FIG. 1 shows a polymeric film layer 10 having active agent 12 disposed thereon. The active agent may be supported substantially on the surface of the polymeric film layer, or it may be disposed within the polymeric film layer to varying extents. Absorption of the active agent may depend on various factors, including the physical characteristics of the active agent and the polymeric material, the application method, temperature of application, etc. FIG. 2 shows a polymeric film layer 10 having active agent 14 applied in more discrete locations than what was shown in FIG. 1. It should be noted that the active agent, though applied to only a portion of the polymeric film layer, may be applied in various patterns and configurations, preferably by a printing process such as ink-jet printing. The implementation of a micro-pipette can be used in accordance with other embodiments of the present invention.

The active agent may be applied to the polymeric film layer by a number of conceivable methods, such as liquids, powders, pastes or gels, dispersed solids, etc. For example, in one aspect the active agent may be formulated and applied to the polymeric film layer as a liquid. In one embodiment, the technology used to print ink onto paper may be adapted to apply an active agent to the polymeric film. Such application systems are highly refined and can be used in high volume industrial applications and/or low volume personal applications. Highly developed printing methods can be adapted to fabricate and control drug production in a very reproducible and high speed process. Furthermore, it should be understood that advances in ink-jet printing technology may be utilized to precisely apply an active agent to a polymeric film layer, thereby enhancing control of the application of the active agent. Ink-jetting of the active agent onto the polymeric film layer can be accomplished by any type of dispensing process known, including thermal and piezoelectric printing. When printing an active agent using ink-jet technology, typically the active agent is carried by a liquid carrier of some type to form a liquid active agent formulations, though this is not necessarily required for certain liquid active agents.

Liquid active agent formulations can comprise an active agent in a pharmaceutically acceptable carrier. Such formulations may remain in the liquid state within the multilayer structure, or they may be at least partially dried or gelled during manufacture. Non-limiting types of excipients that can be used with the present invention include surfactants, polymers, binders, disintegrants, lubricants, antioxidants, preservatives, enteric coatings, release modifiers, colorants, taste masking agents and combinations thereof. In addition, other agents may be included in the formulation to assist in the jettability of the formulation. Such agents may include surfactants, viscosity modifiers, dyes, etc.

Useful organic solvents in which the active agent can be dissolved can include, without limitation, alcohols, chlorinated solvents, ketones, and combinations thereof. Specific examples of useful alcohols include, without limitation, hexanol, pentanol, butanol, propanol, ethanol, methanol, and combinations thereof. Specific examples of useful chlorinated solvents include, without limitation, chloroform, methylene chloride, and combinations thereof. A specific example of a useful ketone can include, without limitation, acetone. One specific embodiment of the present invention includes a solvent system where the organic solvent includes chloroform, ethanol, methanol, acetone, acetonitrile, and combinations thereof. In accordance with some embodiments, these solvents can be removed by drying or evaporation, etc., after applying the active agent to the polymeric film.

It is noted that ink-jet printing, electrostatic printing, ultrasonic printing, etc., can be used in conjunction with the ink-jet printing methods of the present invention to print biocompatible images onto the polymeric material as well. Such printing may include brands, barcodes, or other identifying features. In one aspect, the inks may be visible inks. In another aspect, the inks can be configured such that printing is not visible without the aid of an ultraviolet or infrared light source. Thus, even at the time of dosing a drug delivery system (or at a different time) in accordance with embodiments of the present invention, biocompatible inks can be printed on the device to provide branding, instructions, or the like.

Various active agents are contemplated for inclusion in the pharmaceutical compositions according to various aspects of the present invention. Active agents may include therapeutic, prophylactic, diagnostic, or other known agents. Non-limiting examples of active agents may include amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, mixtures, and combinations thereof.

Though numerous polymeric materials are contemplated for the construction of the polymeric film layers, such materials may include, without limitation, hydroxylpropylmethylcellulose, hydroxylpropylcellulose, carboxymethylcellulose, ethylcellulose, hydroxyethylcellulose, high molecular weight polyethylene oxide, polyvinyl alcohol, polymethylvinylether, poly-(2-ethyl 2-oxazoline), polysaccharides, sodium alginate, polymethacrylates, polyvinylpyrrolidone, polyacrylic acid, polyesters, polylactic acid, poly(lactide-co-glycolide), polycaprolactone, polyglycolic acid, natural or chemically modified starches, polyethylene glycol, polyethylene oxide, 2-methyloxirane, oxirane, glycerin based materials, gelatin, wheat gluten, pectin, xanthan gum, guar gum, algin, chitosan, pullulan, sorbitol, seaweed, milk proteins, rice paper, potato wafer sheets, restructured fruits and vegetables, and combinations thereof. In one specific aspect, the polymeric material may include a water soluble polysaccharide such as pullulan. In another specific aspect, the polymeric material may include polyvinylpyrrolidone.

FIG. 3 shows a plurality of polymeric film layers 10 having applied active agent 14 that are stacked into a multilayer structure 16. In one aspect, the active agent may be applied to the polymeric film layers that have been pre-cut for assembly into the multilayer structure. Alternatively, the active agent may be applied to a polymeric film layer that is then cut prior to assembly into the multilayer structure. In still other embodiments, the multilayer structure can be assembled and cut as a whole. Additionally, the multilayer structure may optionally be capped with a polymeric top layer 18 that lacks active agent. Alternatively, the top layer may contain active agent. Following stacking of the plurality of polymeric film layers, the multilayer structure 16 is heat sealed as shown in FIG. 4. Note that in one embodiment, the heat sealed portion 20 of the multilayer structure in this embodiment does not contain active agent, although such a configuration is not excluded herein. Degradation of the active agent due to heat may be avoided by excluding active agent from the heat sealed portion of the multilayer structure. In an alternative aspect of the present invention, the plurality of polymeric film layers may be joined together into a multilayer structure with a biocompatible adhesive.

It is contemplated that the formulations according to aspects of the present invention can be formulated into various dosage forms. In one aspect, for example, the formulation can be an oral dosage form. As such, in one aspect the formulation may be administered by merely swallowing the multilayer structure. In another aspect, the multilayer structure can be administered in a capsule or other protective coating. These encapsulated dosage forms can be further coated with a polymeric or other art-known coating material to achieve, for example, greater stability on the shelf or in the gastrointestinal tract, or to achieve additional control over drug release. Such coating techniques and materials used therein are well-known in the art. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers, etc., can be used to achieve a coating that can further enhance the sustained release properties of the formulation.

In some aspects, the oral dosage form may include adjuvants such as opacifiers, bulking agents, sweeteners, stabilizing agents, etc. Examples of opacifiers may include, without limitation, titanium dioxide, Talc, calcium carbonate, behenic acid, and cetyl alcohol. Examples of bulking agents may include, without limitation, starch, microcrystalline cellulose, calcium sulfate, calcium phosphate, and lactose. Non-limiting examples of sweeteners may include aspartame, saccharin, sodium cyclamate and Xylitol. Examples of stabilizing agents may include alginic acid glycerylmonostearate, hydroxypropyl cellulose, magnesium, aluminum silicate, and propylene glycol.

The formulations according to aspects of the present invention may also be formulated as transdermal dosage forms. As such, additional layers may be included with controlled release formulations. For example, transdermal formulations may further include backing material preclude release of active agent through the surfaces of the multilayer structure that face away from the skin. The material chosen for the backing should be compatible with the polymer film layers, active agent, and other components such as an enhancer, and should be minimally permeable to any components of the transdermal formulation. In one aspect, the backing may be opaque to protect components of the formulation from degradation from exposure to ultraviolet light. In another aspect, the backing may be transparent in order to minimize the visibility of the patch when applied. Furthermore, the backing should be capable of binding to and supporting the multilayer structure, yet should be pliable enough to accommodate the movements of a person using the transdermal formulation. Suitable materials for the backing include, but are not limited to: metal foils, metallized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene.

Additionally, pressure sensitive adhesive layers may be included for fixing the device against the skin. Such adhesives must be physically and chemically compatible with the active agent and any optional enhancer present. In one aspect of the invention, the adhesives of the transdermal formulation can include polymeric adhesives. Example of such adhesives can include without limitation, acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers or mixtures thereof. In yet another aspect of the invention, contact adhesives for use in the transdermal formulation are acrylic adhesives. Those of ordinary skill in the art will appreciate that the specific type and amount of adhesive polymer used may be selected depending upon the desired specific characteristics of the final product.

The transdermal formulations according to aspects of the present invention can also include one or more of a number of other additives, such as diluents, excipients, emollients, plasticizers, skin irritation reducing agents, or a mixture thereof. Such materials are pharmaceutically acceptable in that they are nontoxic, do not hinder drug delivery, and are not for any other reasons biologically or otherwise undesirable. Examples of such additional materials include water, mineral oils, silicone, inorganic gels, aqueous emulsions, liquid sugars, waxes, petroleum jellies, plasticizers, low molecular weight polymers, and a variety of other oils and polymeric materials. These types of components, as well as others not specifically recited, are well known in the art for inclusion in various transdermal formulations, and may be added as desired to the transdermal drug delivery system of the present invention in specific types and amounts in order to achieve a desired result. Additionally, many transdermal drug delivery formulations have a tendency to cause skin irritation after prolonged exposure to the skin, thus addition of a skin irritation reducing agent aids in achieving a composition that is better tolerated by the skin.

As described herein, the transdermal formulations of the present invention may also optionally include a permeation enhancer, or mixture of permeation enhancers. Useful permeation enhancers may include, without limitation, fatty acids, fatty acid esters, fatty alcohols, fatty acid esters of lactic acid or glycolic acid, glycerol tri-, di-, and monoesters, triacetin, short chain alcohols, and mixtures thereof. One skilled in the art would, however, understand that many other enhancers could be utilized in these formulations, and would thus be included within the scope of the present invention.

The formulations according to aspects of the present invention can also be formulated as transmucosal dosage forms. Transmucosal dosage forms may include, without limitation, buccal, vaginal, and anal preparations. Many of the abovementioned components may be utilized in various transmucosal dosage forms, as would be recognized by one of ordinary skill in the art once in possession of the present disclosure. However, in one aspect an adhesive layer may be included in the transmucosal dosage form to maintain the position of the multilayer structure during use. For example, an adhesive layer on one side of a buccal patch can hold the dosage form fixed in the buccal cavity during the release of the active agent, whether controlled release or immediate release, as is known in the art.

### EXAMPLES

The following examples are given to illustrate embodiments of the present invention. It should be understood, however, that the invention is not to be limited to the specific conditions or details described in these examples.

### Example 1

Glyburide is completely dissolved in a solution of choloroform (32.2 vol %), ethanol (38.8 vol %), and water (29 vol %) at a concentration of 3.3 mg/mL to form a drug solution. The drug solution is then ink-jetted using piezoelectric printhead in discrete locations onto a film of pullulan where the chloroform and ethanol are allowed to evaporate. The pullulan is cut into 2 cm×2 cm squares and heat sealed into various multilayer structures, including a two layer structure, a three layer structure, and a four layer structure.

### Example 2

Glyburide is completely dissolved in a solution of ethanol (65 vol %) and water (35 vol %) at a concentration of 3.3 mg/mL to form a drug solution. The drug solution is then ink-jetted using piezoelectric printhead in discrete locations onto a film of pullulan where the chloroform and ethanol are allowed to evaporate. The pullulan is cut into 2 cm×2 cm squares and heat sealed into various multilayer structures, including a two layer structure, a three layer structure, and a four layer structure.

### Example 3

Glyburide is completely dissolved in a solution of acetone (65 vol %) and water (35 vol %) at a concentration of 3.3 mg/mL to form a drug solution. The drug solution is then ink-jetted using piezoelectric printhead onto a film of pullulan and the acetone is allowed to evaporate. The pullulan is cut into 2 cm×2 cm squares and heat sealed into various multilayer structures, including a two layer structure, a three layer structure, and a four layer structure.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A pharmaceutical composition, comprising:
a plurality of polymeric film layers heat sealed together as a multilayer structure; and
controlled amounts of active agent dissolved or dispersed in a liquid vehicle which have been ink-jetted on at least one of the plurality of polymeric film layers prior to being heat sealed together, wherein the active agent is within the multilayer structure.

2. The composition of claim 1, wherein the plurality of polymeric film layers include a member selected from hydroxylpropylmethylcellulose, hydroxylpropylcellulose, carboxymethylcellulose, ethylcellulose, hydroxyethylcellulose, high molecular weight polyethylene oxide, polyvinyl alcohol, polymethylvinylether, poly-(2-ethyl 2-oxazoline), polysaccharides, sodium alginate, polymethacrylates, polyvinylpyrrolidone, polyacrylic acid, polyesters, polylactic acid, poly(lactide-co-glycolide), polycaprolactone, polyglycolic acid, natural or chemically modified starches, polyethylene glycol, polyethylene oxide, 2-methyloxirane, oxirane, glycerin based materials, gelatin, wheat gluten, pectin, xanthan gum, guar gum, algin, chitosan, pullulan, sorbitol, seaweed, milk proteins, rice paper, potato wafer sheets, restructured fruits and vegetables, and combinations thereof.

3. The composition of claim 1, wherein the plurality of polymeric film layers includes pullulan.

4. The composition of claim 1, wherein the plurality of polymeric film layers includes polyvinylpyrrolidone.

5. The composition of claim 1, wherein the active agent is dispersed within only a portion of the multilayer structure.

6. The composition of claim 1, wherein the active agent includes a member selected from amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, mixtures, and combinations thereof.

7. The composition of claim 1, wherein the active agent is located substantially between adjacent polymeric film layers.

8. The composition of claim 1, wherein at least a portion of the active agent is absorbed into at least one of the plurality of polymeric film layers.

9. The composition of claim 1, wherein the pharmaceutical composition is formulated as a transdermal or transmucosal dosage form.

10. The composition of claim 1, wherein the pharmaceutical composition is formulated as an oral dosage form.

11. The composition of claim 1, wherein active agent is absent in those regions of the multilayer structure having polymeric film layers heat sealed directly together.

12. The composition of claim 1, wherein the active agent is dissolved in the liquid vehicle.

13. The composition of claim 12, wherein upon jetting the dissolved active agent on the at least one of the plurality of polymeric film layers, the active agent forms nanoparticles.

14. The composition of claim 1, wherein the plurality of polymeric film layers includes at least three layers of the same material.

15. The composition of claim 1, wherein the active agent is precisely printed via control from a digital source to generate a predetermined pattern of active agent within the multilayer structure.

16. The composition of claim 1, in controlled release form.

17. The composition of claim 1, in an immediate release form.

18. A method of making a pharmaceutical composition, comprising:
dispersing or dissolving an active agent in a liquid vehicle to form an ink-jettable composition;
precisely ink-jetting the ink-jettable composition on at least one of a plurality of polymeric film layers; and
heat sealing the plurality of polymeric film layers into a multilayer structure.

19. The method of claim 18, wherein the active agent is dissolved in the liquid vehicle.

20. The method of claim 19, wherein after ink-jetting the ink-jettable composition on the at least one of the plurality of polymeric film layers, the active agent comes out of solution and forms nanoparticles.

21. The method of claim 18, wherein the step of ink-jetting the ink-jettable composition includes avoiding printing in areas where heat sealing occurs.

22. The method of claim 18, wherein the step of printing is by a thermal ink-jet printing process.

23. The method of claim 18, wherein the step of printing is by a piezo ink-jet printing process.

24. The method of claim 18, wherein the active agent includes a member selected from amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, mixtures, and combinations thereof.

25. The method of claim 18, wherein the plurality of polymeric film layers include a member selected from hydroxylpropylmethylcellulose, hydroxylpropylcellulose, carboxymethylcellulose, ethylcellulose, hydroxyethylcellulose, high molecular weight polyethylene oxide, polyvinyl alcohol, polymethylvinylether, poly-(2-ethyl 2-oxazoline), polysaccharides, sodium alginate, polymethacrylates, polyvinylpyrrolidone, polyacrylic acid, polyesters, polylactic acid, poly(lactide-co-glycolide), polycaprolactone, polyglycolic acid, natural or chemically modified starches, polyethylene glycol, polyethylene oxide, 2-methyloxirane, oxirane, glycerin based materials, gelatin, wheat gluten, pectin, xanthan gum, guar gum, algin, chitosan, pullulan, sorbitol, seaweed, milk proteins, rice paper, potato wafer sheets, restructured fruits and vegetables, and combinations thereof.

26. The method of claim 18, wherein the plurality of polymeric film layers includes pullulan.

27. The method of claim 18, wherein the plurality of polymeric film layers includes polyvinylpyrrolidone.

28. The method of claim 18, wherein the pharmaceutical composition is formulated in a controlled release form.

29. The method of claim 18, wherein the pharmaceutical composition is formulated in an immediate release form.

30. A method of using a pharmaceutical composition, comprising administering to a subject a pharmaceutical composition including:
a plurality of polymeric film layers heat sealed together as a multilayer structure; and
controlled amounts of active agent dissolved or dispersed in a liquid vehicle which have been ink-jetted on at least one of the plurality of polymeric film layers prior to being heat sealed together, wherein the active agent is within the multilayer structure.

31. The method of claim 30, wherein the pharmaceutical composition is administered to the subject as a transdermal dosage form.

32. The method of claim 30, wherein the pharmaceutical composition is administered to the subject as an oral dosage form.

33. The method of claim 30, wherein the pharmaceutical composition is administered to the subject as a transmucosal dosage form.

34. The method of claim 30, wherein the active agent is selected from amiodarone HCL, atorvastatin, candesartan, carvedilol, clopidogrel bisulfate, dipyridamole, eprosartan mensylate, felodipine, furosemide, isradipine, lovastatin, metolazone, propafenone HCL, quinapril, ramipril, simvastatin, trandolapril, valsartan, clozapine, entacapone, fluphenazine, fluvoxamine, imipramine, olanzapine, paroxetine, sertraline, triazolam, zaleplon, ziprasidone, acyclovir, amphotericin B, amprenavir, cefdinir, cefixime, ceftazidime, clarithromycin, didanosine, efavirenz, ganciclovir, itraconazole, melfloquine, norfloxacin, nystatin, ritonavir, saquinavir, tenofovir disoproxil fumarate, beclomethasone dipropionate, bosentan, budesonide, fexofenadine, flunisolide, fluticasone, loratadine, mometasone, salmeterol xinafoate, triamcinolone acetonide, zafirlukast, celecoxib, diclofenac sodium, dihydroergotamine mesylate, ergoloid mesylates, ergotamine tartrate, fentanyl citrate, nabumetone, azathioprine, carboplatin, cisplatin, cyclosporine, docetaxel, etoposide, flurouracil, irinotecan, letrozole, melphalan, mitotane, paclitaxel, pimecrolimus, sirolimus, tacrolimus, valrubicin, ethinyl estradiol, danazol, follotropin beta, medroxy-progesterone, methyl-testosterone, raloxifene HCL, sildenafil citrate, testosterone, calcitrol, dronabinol, famotidine, glyburide, isotretinoin, megestrol, modafinil, nimodipine, pioglitazone, propofol, thalidomide, betamethasone, triamcinolone, piroxicam, glimepiride, glipizide, digoxin, prednisolone, indomethacine, nadolol, fluconazol, cisapride, ibuprofen, acetaminophen, carbamazepine, nifedipine, ketoprofen, and derivatives, prodrugs, mixtures, and combinations thereof.

35. The method of claim 30, wherein the plurality of polymeric film layers include a member selected from hydroxylpropylmethylcellulose, hydroxylpropylcellulose, carboxymethylcellulose, ethylcellulose, hydroxyethylcellulose, high molecular weight polyethylene oxide, polyvinyl alcohol, polymethylvinylether, poly-(2-ethyl 2-oxazoline), polysaccharides, sodium alginate, polymethacrylates, polyvinylpyrrolidone, polyacrylic acid, polyesters, polylactic acid, poly(lactide-co-glycolide), polycaprolactone, polyglycolic acid, natural or chemically modified starches, polyethylene glycol, polyethylene oxide, 2-methyloxirane, oxirane, glycerin based materials, gelatin, wheat gluten, pectin, xanthan gum, guar gum, algin, chitosan, pullulan, sorbitol, seaweed, milk proteins, rice paper, potato wafer sheets, restructured fruits and vegetables, and combinations thereof.

36. The method of claim 30, wherein active agent is absent in those regions of the multilayer structure having polymeric film layers heat sealed directly together.

37. The method of claim 30, wherein the active agent is dissolved in the liquid vehicle.

38. The method of claim 37, wherein upon jetting the dissolved active agent on the at least one of the plurality of polymeric film layers, the active agent forms nanoparticles.

39. The method of claim 30, wherein the plurality of polymeric film layers includes at least three layers of the same material.

40. A system for making a pharmaceutical composition, comprising:
an ink-jetting means for digitally applying an active agent to only a portion of at least one of a plurality of polymeric film layers; and
a heat sealing means for bonding the plurality of polymeric film layers into a multilayer structure such that the heat sealing occurs only where no active agent is present in the multilayer structure.
